(19) United States
(12) Patent Application Publication
(10) Pub. No.: US 2026/0069451 A1
(43) Pub. Date: Mar. 12, 2026

(54) Cold-hot Apparatus with Hidden Ring Pull

(71) Applicant: Lufeng Automatic Control Equipment Co., Ltd., Beijing (CN)

(72) Inventor: Houqi Hu, Beijing (CN)

(21) Appl. No.: 18/914,258

(22) Filed: Oct. 14, 2024

(30) Foreign Application Priority Data
Sep. 6, 2024 (CN) 202422193699.X

(51) Int. Cl. A61F 7/00; F16B 45/00

(52) U.S. Cl. CPC A61F 7/0085; F16B 45/002; A61F 2007/006

(57) ABSTRACT

The present application relates to the field of medical care equipment, and in particular to a cold-hot apparatus with a hidden ring pull, wherein the cold-hot apparatus comprises a housing, a cold-hot assembly and a ring pull, wherein the housing is internally provided with the cold-hot assembly, and both sides of the housing are rotatably provided with the ring pull; the two sides of the housing are provided with fixing grooves corresponding to the ring pull, which are adapted thereto; the ring pull of the cold-hot apparatus is used for connecting with the strap to fix the cold-hot apparatus to the nursing part; through the rotatable arrangement of the ring pull, after the strap is bound, the angle of the adapted nursing part can be adjusted by rotating the ring pull, the ring pull can be received in the fixing groove to reduce the overall volume and facilitate carrying.

### CROSS REFERENCE TO RELATED APPLICATIONS

This application claims priority to Chinese Patent Application No. 202422193699.X, filed on Sep. 6, 2024, the content of which is incorporated herein by reference in its entirety.

### TECHNICAL FIELD

The present application relates to the field of medical care equipment, and more particularly to a cold-hot apparatus with a hidden ring pull.

### BACKGROUND

The cold-hot apparatus can perform cold or hot compress on the nursing part through the built-in semiconductor cold-hot sheet to play the role of physical therapy. However, in designing a portable cold-hot apparatus, a ring pull structure is usually provided on the outside of the housing, so that the cold-hot apparatus can be attached and fixed to by the connection between the ring pull and a strap. The common ring pull structure is mostly a structure fixedly assembled on the outer side of the housing, which occupies a certain storage volume, and has a certain limit because it cannot be folded or rotated during use.

### SUMMARY

The technical problem to be solved by the present application is to provide a cold-hot apparatus with a hidden ring pull which can be conveniently used and has a reduced storage volume.

In order to solve the above-mentioned technical problem, the technical solution adopted by the present application is: a cold-hot apparatus with a hidden ring pull, which comprises a housing, a cold-hot assembly and a ring pull, wherein the housing is internally provided with the cold-hot assembly, and both sides of the housing are rotatably provided with the ring pull; the two sides of the housing are provided with fixing grooves corresponding to the ring pull, which are adapted thereto.

Further, the ring pull is detachably connected to the fixing grooves.

Further, a snap fit groove is provided in the fixing groove, and the ring pull and a side of the fixing groove provided with the snap fit groove are respectively provided with a snap fit point cooperating therewith.

Further, a notch is provided in the middle of one side edge of the fixing groove, and a protruding pulling block is provided on the ring pull, and the pulling block is pressed into the notch.

Further, one face of the housing is a working face, and the cold-hot assembly is provided at the working face; a vibration motor is provided within the housing and abuts against the working face.

Further, the apparatus further comprises a fan provided within the housing; an upper side of the housing is provided with an air inlet, and an air outlet is provided at a side of the housing; an air guide lid is provided between the fan and the air inlet, and the middle part of the air guide lid is in communication with the fan; the air guide lid has a disc shape, and an arc-shaped connecting surface is provided between the edge and the middle part thereof.

Further, the apparatus further comprises a battery, wherein the battery is provided between the side of the cold-hot assembly and the inner wall of the housing, the battery is electrically connected to the cold-hot assembly, and the battery is a soft-pack battery.

Further, the apparatus further comprises a radiator provided between the fan and the cold-hot assembly.

Further, the housing includes an upper case and a lower case, wherein the upper case is fastened to the lower case; a first mounting rack extends toward the lower case inside the upper case, a second mounting rack extends toward the upper case inside the lower case, a fan is provided in the first mounting rack, and a radiator is provided in the second mounting rack.

Further, a battery is included, and the first and second mounting racks have a space with the inner wall of the upper case and the inner wall of the lower case, and the battery is fitted within the space. The beneficial effects of the present application are: the ring pull of the cold-hot apparatus is used for connecting with the strap to fix the cold-hot apparatus to the nursing part; through the rotatable arrangement of the ring pull, after the strap is bound, the angle of the adapted nursing part can be adjusted through the rotation of the ring pull, and the arrangement that the ring pull can be received in the fixing groove can receive the ring pull to reduce the overall volume and facilitate carrying.

### BRIEF DESCRIPTION OF THE DRAWINGS

FIG. 1 is a schematic structural diagram showing a cold-hot apparatus with a hidden ring pull according to an embodiment of the present application;

FIG. 2 is a schematic structural diagram showing an internal structure of a cold-hot apparatus with a hidden ring pull according to an embodiment of the present application; and

FIG. 3 is a schematic structural diagram showing a vibration motor of a cold-hot apparatus with a hidden ring pull inside a housing according to an embodiment of the present application.

### Description of Reference Numerals

1. housing;
11. upper case; 111. first mounting rack; 112. air inlet;
12. lower case; 121. second mounting rack; 122. air outlet; 123. fixing groove; 124. snap fit groove; 125. notch;
2. semiconductor cold-hot sheet; 3. cold-hot conductor; 4. air guide lid; 5. fan; 6. radiator; 7. ring pull; 71. snap fit point; 72. pulling block; 8. battery; 9. vibration motor.

### DETAILED DESCRIPTION OF THE EMBODIMENTS

In order to explain the technical contents, the objects, and the effects of the present application in detail, the embodiments will be described below with reference to the accompanying drawings.

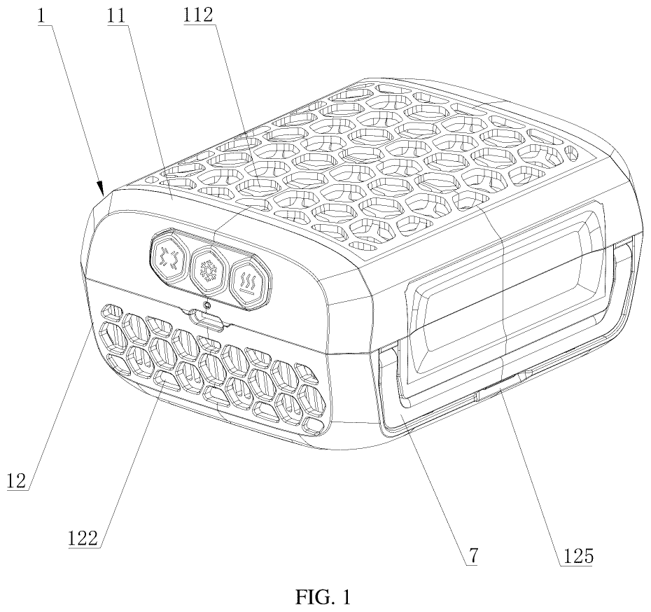

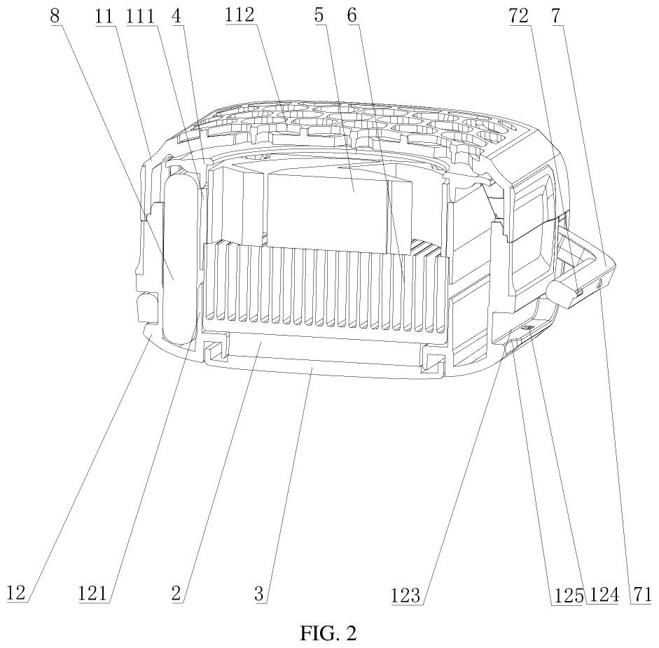

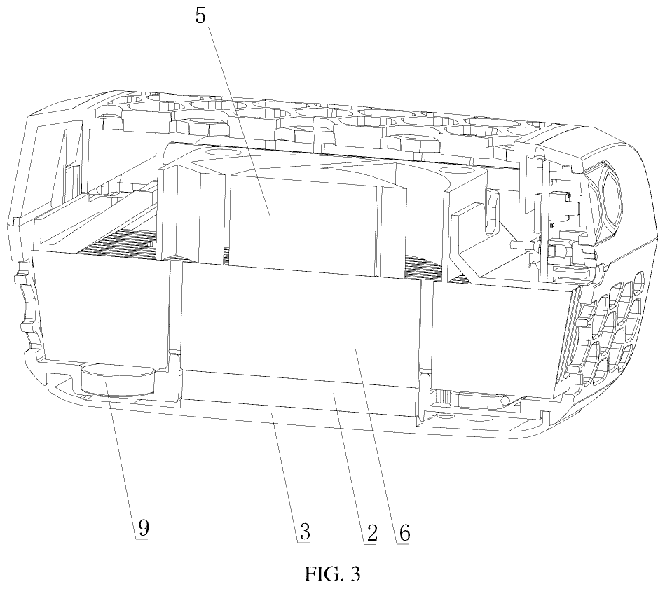

With reference to FIGS. 1 to 3, a cold-hot apparatus with a hidden ring pull, which comprises a housing 1, a cold-hot assembly and a ring pull 7, wherein the housing 1 is internally provided with the cold-hot assembly, and both sides of the housing 1 are rotatably provided with the ring pull 7; the two sides of the housing 1 are provided with fixing grooves 123 corresponding to the ring pull 7, which are adapted thereto.

It can be seen from the above description that the beneficial effects of the present application are: the ring pull 7 of the cold-hot apparatus is used for connecting with the strap to fix the cold-hot apparatus to the nursing part; through the rotatable arrangement of the ring pull 7, after the strap is bound, the angle of the adapted nursing part can be adjusted through the rotation of the ring pull 7, and the arrangement that the ring pull 7 can be received in the fixing groove 123 can receive the ring pull 7 to reduce the overall volume and facilitate carrying.

Further, the ring pull 7 is detachably connected to the fixing grooves 123.

It can be seen from the above-mentioned description that by the detachable connection arrangement of the ring pull 7 and the fixing groove 123, the ring pull 7 is prevented from rotating and falling out of the fixing groove 123 due to shaking or the like when being received.

Further, a snap fit groove 124 is provided in the fixing groove 123, and the ring pull 7 and a side of the fixing groove 123 provided with the snap fit groove 124 are respectively provided with a snap fit point 71 cooperating therewith.

It can be seen from the above-mentioned description that the snap fit between the ring pull 7 and the fixing groove 123 is achieved by the snap fit between the snap fit groove 124 in the fixing groove 123 and the snap fit point 71 on the ring pull 7.

Further, a notch 125 is provided in the middle of one side edge of the fixing groove 123, and a protruding pulling block 72 is provided on the ring pull 7, and the pulling block 72 is pressed into the notch 125.

As can be seen from the above description, by providing a clearance space through the notch 125 of the fixing groove 123, the user can lift the pulling block 72 at the notch 125 to rotate the ring pull 7 out of the fixing groove 123 when the ring pull 7 needs to be lifted.

Further, one face of the housing 1 is a working face, and the cold-hot assembly is provided at the working face; a vibration motor 9 is provided inside the housing 1, and the vibration motor 9 abuts against the working face.

It can be seen from the above description that the vibration function of the apparatus is achieved by the arrangement of the vibration motor 9 against the working face.

Further, the apparatus further comprises a fan 5 provided within the housing 1; an upper side of the housing 1 is provided with an air inlet 112, and an air outlet 122 is provided at a side of the housing 1; an air guide lid 4 is provided between the fan 5 and the air inlet 112, and the middle part of the air guide lid 4 is in communication with the fan 5; the air guide lid 4 has a disc shape, and an arc-shaped connecting surface is provided between the edge and the middle part thereof.

It can be seen from the above-mentioned description that the cold-hot assembly is cooled by the fan 5, and external air is brought into the interior of the housing 1 from the air inlet 112 at the upper side of the housing 1 by the fan 5 to exchange heat with the cold-hot assembly and then discharged from the air outlet 122 at the side of the housing 1; through the arrangement of the arc-shaped connecting surface of the air guide lid 4, the air drawn in at the air inlet 112 can be quickly collected in the middle part of the air guide lid 4 under the guidance of the arc-shaped connecting surface and quickly exchange heat with the cold-hot assembly via the fan 5.

Further, the apparatus further comprises a battery 8, wherein the battery 8 is provided between the side of the cold-hot assembly and the inner wall of the housing 1, the battery 8 is electrically connected to the cold-hot assembly, and the battery 8 is a soft-pack battery 8.

It can be seen from the above-mentioned description that the soft-pack battery 8 is designed between the side of the cold-hot assembly and the inner wall of the housing 1, and the space between the cold-hot assembly and the housing 1 is filled by using the flat structure of the soft-pack battery 8, so that the structure is more compact, the volume of the apparatus can be effectively reduced, and the large space occupation of the conventional cylindrical battery 8 is reduced.

Further, the apparatus further comprises a radiator 6 provided between the fan 5 and the cold-hot assembly.

It can be seen from the above description that heat exchange over a larger area is achieved by the radiator 6, and heat dissipation efficiency is accelerated.

Further, the housing 1 includes an upper case 11 and a lower case 12, wherein the upper case 11 is fastened to the lower case 12; a first mounting rack 111 extends toward the lower case 12 inside the upper case 11, a second mounting rack 121 extends toward the upper case 11 inside the lower case 12, a fan 5 is provided in the first mounting rack 111, and a radiator 6 is provided in the second mounting rack 121.

It can be seen from the above-mentioned description that the housing 1 adopts a combination form in which the upper case 11 and the lower case 12 are snap-fit; the first mounting rack 111 of the upper case 11 and the second mounting rack 121 of the lower case 12 fix the fan 5 and the radiator 6, respectively, to form a compact mounting structure.

Further, a battery 8 is included, and the first and second mounting racks 111 and 121 have a space with the inner wall of the upper case 11 and the inner wall of the lower case 12, and the battery 8 is fitted within the space.

It can be seen from the above-mentioned description that the first mounting rack 111 and the second mounting rack 121 not only fix the structure of the tailstock fan 5 and the radiator 6, but also cooperate with the inner wall of the housing 1 to form an assembly space of the battery 8, and the structure space is rationally arranged to make the apparatus compact.

### Embodiment I

As shown in FIG. 1 to FIG. 3, the cold-hot apparatus with a hidden ring pull of the present embodiment includes a housing 1, a cold-hot assembly, a fan 5, a radiator 6, a battery 8, a circuit assembly, a vibration motor 9, and a ring pull 7.

Further, the housing 1 includes an upper case 11 and a lower case 12, wherein the upper case 11 is fastened to the lower case 12; an air inlet 112 is provided at the top of the upper case 11, a first mounting rack 111 extends from the inside of the upper case 11 towards the lower case 12, a second mounting rack 121 extends toward the upper case 11 inside the lower case 12, a fan 5 is provided in the first mounting rack 111, and a radiator 6 is provided in the second mounting rack 121. An air guide lid 4 is provided between the upper case 11 and the fan 5, and the middle part of the air guide lid 4 is in communication with the fan 5; the air guide lid 4 is in the shape of a disk, and has an arc-shaped connecting surface between the edge and the middle thereof; one side of the air guide lid 4 adjacent to the fan 5 is provided with a snap fit block fixedly snap fit to an edge of the fan 5;

An air outlet 122 is provided on a side surface of the lower case 12, a contact port is provided at the bottom of the lower case 12, the cold-hot assembly comprises a semiconductor cold-hot sheet 2 and a cold-hot conductor 3, and the cold-hot conductor 3 is snap fit to the contact port to form a working face as a contact structure with a nursing part; the vibration motor 9 is provided in the lower case 12 and abuts against the cold-hot conductor 3; and a semiconductor cold-hot sheet 2 is provided between the radiator 6 and the cold-hot conductor 3.

The first and second mounting racks 111 and 121 have a space with the inner wall of the upper case 11 and the inner wall of the lower case 12, and the battery 8 is fitted within the space. The battery 8 is a soft-pack battery 8 having a flat structure. In particular, a circuit assembly for adjusting the function of the apparatus is also provided in the space. The circuit assembly is electrically connected to the battery 8, the cold-hot assembly and the fan 5.

Both sides of the lower case 12 are rotatably provided with ring pulls 7, and both sides of the housing 1 are provided with fixing grooves 123 corresponding to the ring pulls 7. A snap fit groove 124 is provided in the fixing groove 123, and the ring pull 7 and the side of the fixing groove 123 provided with the snap fit groove 124 are provided with a snap fit point 71 cooperating therewith; a notch 125 is provided in the middle of one side edge of the fixing groove 123, and a protruding pulling block 72 is provided on the ring pull 7, and the pulling block 72 is pressed into the notch 125.

Specifically, the housing 1 has an overall rectangular shape, the lower case 12 has four side surfaces, the ring pull 7 is provided on the side surface of the lower case 12 parallel to the length direction thereof, and the air outlet 122 is provided on the side surface of the lower case 12 parallel to the width direction thereof. The circuit assembly is provided with a charging port and an operation button on the side of the upper case 11 on the same side as the air outlet 122.

The edges of the side surface of the upper case 11 provided with the air inlet 112 and the side surface of the lower case 12 provided with the air outlet 122 both have a curved surface, and the design of the curved surface enables the air inlet 112 or the air outlet 122 at the curved surface to ensure the heat dissipation of the apparatus even if the side surface of the upper case 11 and the side surface of the lower case 12 are in parallel contact with the nursing part.

In summary, the present application provides a cold-hot apparatus with a hidden ring pull, wherein the ring pull of the cold-hot apparatus is used for connecting with the strap to fix the cold-hot apparatus to the nursing part; through the rotatable arrangement of the ring pull, after the strap is bound, the angle of the adapted nursing part can be adjusted through the rotation of the ring pull, and the arrangement that the ring pull can be received in the fixing groove can receive the ring pull to reduce the overall volume and facilitate carrying.

The above-mentioned embodiments are merely examples of the present application and are not intended to limit the scope of the present application, and all changes which come within the meaning and range of equivalence of the present application and the appended claims are to be embraced within their scope.

1. A cold-hot apparatus with a hidden ring pull, comprising a housing, a cold-hot assembly and a ring pull, wherein the housing is internally provided with the cold-hot assembly, and both sides of the housing are rotatably provided with the ring pull; the two sides of the housing are provided with fixing grooves corresponding to the ring pull, which are adapted thereto.

2. The cold-hot apparatus with a hidden ring pull according to claim 1, wherein the ring pull is detachably connected to the fixing groove.

3. The cold-hot apparatus with a hidden ring pull according to claim 2, wherein a snap fit groove is provided in the fixing groove, and the ring pull and a side of the fixing groove provided with the snap fit groove are respectively provided with a snap fit point cooperating therewith.

4. The cold-hot apparatus with a hidden ring pull according to claim 1, wherein a notch is provided in the middle of one side edge of the fixing groove, and a protruding pulling block is provided on the ring pull, and the pulling block is pressed into the notch.

5. The cold-hot apparatus with a hidden ring pull according to claim 1, wherein one face of the housing is a working face, and the cold-hot assembly is provided at the working face; a vibration motor is provided within the housing and abuts against the working face.

6. The cold-hot apparatus with a hidden ring pull according to claim 1, further comprising a fan provided within the housing; an upper side of the housing is provided with an air inlet, and an air outlet is provided at a side of the housing; an air guide lid is provided between the fan and the air inlet, and the middle part of the air guide lid is in communication with the fan; the air guide lid has a disc shape, and an arc-shaped connecting surface is provided between the edge and the middle part thereof.

7. The cold-hot apparatus with a hidden ring pull according to claim 1, further comprising a battery, wherein the battery is provided between the side of the cold-hot assembly and the inner wall of the housing, the battery is electrically connected to the cold-hot assembly, and the battery is a soft-pack battery.

8. The cold-hot apparatus with a hidden ring pull according to claim 6, further comprising a radiator provided between the fan and the cold-hot assembly.

9. The cold-hot apparatus with a hidden ring pull according to claim 6, the housing includes upper case and lower case, wherein the upper case is fastened to the lower case; a first mounting rack extends toward the lower case inside the upper case, a second mounting rack extends toward the upper case inside the lower case, a fan is provided in the first mounting rack, and a radiator is provided in the second mounting rack.

10. The cold-hot apparatus with a hidden ring pull according to claim 9, further comprising a battery, wherein the first and second mounting racks have a space with the inner wall of the upper case and the inner wall of the lower case, and the battery is fitted within the space.
